(19) United States
(12) Patent Application Publication
(10) Pub. No.: US 2024/0091243 A1
(43) Pub. Date: Mar. 21, 2024

(54) METHODS FOR PREVENTING VIRAL INFECTION

(71) Applicants: Ameer E. HASSAN, Harlingen, TX (US); Yousef Hasan Ahmad KHALILI, Dubai (AE)

(72) Inventors: Ameer E. HASSAN, Harlingen, TX (US); Yousef Hasan Ahmad KHALILI, Dubai (AE)

(21) Appl. No.: 18/261,592

(22) PCT Filed: Jan. 29, 2022
(86) PCT No.: PCT/US2022/014456

§ 371 Date: Jul. 14, 2023

Related U.S. Application Data
(60) Provisional application No. 63/143,321, filed on Jan. 29, 2021.

(51) Int. Cl. A61K 31/7008; A61K 31/07; A61K 31/375; A61K 31/519; A61K 31/593; A61K 33/30; A61P 31/14

(52) U.S. Cl. CPC A61K 31/7008; A61K 31/07; A61K 31/375; A61K 31/519; A61K 31/593; A61K 33/30; A61P 31/14

(57) ABSTRACT

The present disclosure relates to compounds and methods for preventing infection, symptoms, or sequelae resulting from viral infection, including influenza infection, rhinovirus infection, or betacoronavirus infection, such as human coronaviruses such as SARS coronaviruses, MERS coronaviruses, and COVID-19, including Acute Respiratory Distress Syndrome (ARDS) associated with the viral infection.

### CROSS-REFERENCE TO RELATED APPLICATIONS

This application claims priority under 35 U.S.C. § 119(e) to U.S. Provisional Application Ser. No. 63/143,321 filed on Jan. 29, 2021, the entire disclosure of which is incorporated herein by reference.

### FIELD

The present disclosure relates to methods for preventing infection, symptoms, or sequelae resulting from viral infection, including influenza infection, rhinovirus infection, or betacoronavirus infection, such as human coronaviruses such as SARS coronaviruses, MERS coronaviruses, and COVID-19, including Acute Respiratory Distress Syndrome (ARDS) associated with the viral infection.

### BACKGROUND

Viral infections represent one of the most prevalent health risks in the human population. Viral infections originate from a variety of viruses including influenza, coronavirus, rhinovirus, norovirus, rotavirus, exanthematous virus, hepatic virus, and the like. The severity of illness resulting from viral infections can range from minimal or mild symptoms to lethal clinical outcomes. Coronaviruses, especially betacoronaviruses, are a group of related RNA viruses that can affect humans and can cause respiratory tract infections that range from mild to lethal. The betacoronavirus that cause human diseases (human coronaviruses, HCoV) include seven members designated as SARS-CoV-1 (SARS), MERS-CoV (MERS), HCoV-HKU1, HCoV-NL63, HCoV-OC43, HCoV-229E, and most recently SARS-CoV-2 (COVID-19). Mild illnesses in humans include some cases of the common cold (which is caused by coronaviruses and is also caused by other viruses, predominantly rhinoviruses), while more lethal varieties can cause SARS, MERS, and COVID-19.

COVID-19 is thought to spread from person to person, mainly through respiratory droplets produced when an infected person breathes, coughs, or sneezes. Emerging data suggests that the severity of COVID-19 may correlate with viral load in the lungs of patients. After inhalation, SARS-CoV-2 infects epithelial cells in the nasal cavity and begins to replicate. Infected cells shed viral particles, which then infect neighboring cells. As the disease progresses, viral particles infect alveolar type II cells in the lung. These cells produce large amounts of viral particles and ultimately die, causing damage to the epithelial lining of the lung. This damage, and the corresponding immunological response, results in a type of pneumonia. As of August 2020, over 25 million people have been infected in at least 200 countries around the world, with most cases being reported in the United States, Brazil, and India, and the worldwide death toll from the virus is quickly approaching 850,000.

The clinical presentation of infection of COVID-19 is primarily manifested as malignant pneumonia. A current list of COVID-19 symptoms identified by the Centers of Disease Control (CDC) include: fever, cough, shortness of breath or difficulty breathing, chills, repeated shaking with chills, muscle pain, headache, sore throat, loss of taste or sense of smell, persistent pain or pressure in the chest, confusion or inability to arouse, bluish lips or face, diarrhea, or vomiting. The severity levels of COVID-19 are generally categorized into three levels: mild illness (generally asymptomatic); severe illness (including measureable breathing difficulties); and critical illness (characterized by respiratory failure, shock, or multi-organ failure). Although the overall mortality rate of COVID-19 is low (1.4-2.3%), patients with comorbidities are more likely to have severe disease and subsequent mortality. Most available studies have shown that diabetes mellitus (DM) is associated with more severe disease, acute respiratory distress syndrome (ARD) and increased mortality.

To date, no effective preventions of SARS-CoV-2 or the resulting illnesses resulting from SARS-CoV-2 infection have been developed or approved. Thus, there is a need for preventions for viral infections, such as influenza and betacoronaviruses, including SARS-CoV-2, including preventions for the diseases and disorders created or exacerbated by the viral infection.

### SUMMARY

In one aspect, the disclosure provides a method of preventing a subject from having a viral infection, such as influenza infection, rhinovirus infection, or betacoronavirus infection, comprising administering a prophylactically effective amount of N-acetyl glucosamine.

In another aspect, the disclosure provides a pharmaceutical composition comprising N-acetyl glucosamine, and optionally a pharmaceutically acceptable carrier or excipient, wherein the N-acetyl glucosamine is in a prophylactically effective amount for preventing a viral infection, such as influenza infection, rhinovirus infection, or betacoronavirus infection.

In another aspect, the disclosure provides a use of N-acetyl glucosamine in the preparation of a medicament for preventing a subject having a viral infection, such as influenza infection, rhinovirus infection, or betacoronavirus infection.

Additional embodiments, features, and advantages of the disclosure will be apparent from the following detailed description and through practice of the disclosure. The compounds, methods, and compositions of the present disclosure can be described as embodiments in any of the following enumerated clauses. It will be understood that any of the embodiments described herein can be used in connection with any other embodiments described herein to the extent that the embodiments do not contradict one another.
1. A method of preventing illness caused by a viral infection, such as influenza infection, rhinovirus infection, or betacoronavirus infection, comprising administering a prophylactically effective amount of N-acetyl glucosamine.
2. The method of clause 1, wherein the betacoronavirus infection that is prevented is SARS-CoV-2 infection.
3. The method of clause 1 or 2, wherein the administering a prophylactically effective amount of N-acetyl glucosamine prevents or reduces the chances of one or more symptoms in the subject, wherein the one or more symptoms is selected form the groups consisting of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.
4. The method of any one of clauses 1 to 3, wherein the prophylactically effective amount prevents one or more symptoms of the betacoronavirus infection.
5. The method of any of one of clauses 1 to 4, wherein the N-acetyl glucosamine is administered intravenously, orally, subcutaneously, buccally, transdermally, or nasally.
6. The method of any of one of clauses 1 to 5, wherein the N-acetyl glucosamine is administered orally.
7. The method of any one of clauses 1 to 6, wherein the prophylactically effective amount of the N-acetyl glucosamine is in the range of about 200 mg to about 2100 mg.
8. The method of any one of clauses 1 to 7, wherein the prophylactically effective amount of the N-acetyl glucosamine is administered once a day (QD), twice a day (BID), or three times a day (TID).
9. The method of any one of clauses 1 to 8, wherein the prophylactically effective amount of the N-acetyl glucosamine is administered twice a day (BID), at a dose of about 300 mg to about 900 mg per dose.
10. The method of any of clauses 1 to 9, further comprising administration of one or more additional supplement agents.
11. The method according to clause 10, wherein the one or more additional supplement agents is a vitamin or an essential mineral.
12. The method according to clause 10 or 11, wherein the one or more additional supplement agents is vitamin A, a B vitamin, vitamin C. vitamin D, or zinc.
13. A pharmaceutical composition comprising N-acetyl glucosamine and optionally a pharmaceutically acceptable carrier or excipient, wherein the N-acetyl glucosamine is in a prophylactically effective amount for preventing a viral infection, such as a betacoronavirus infection.
14. The pharmaceutical composition of clause 13, wherein the N-acetyl glucosamine is in an amount of about 200 mg to about 2100 mg in the composition.
15. The pharmaceutical composition of clause 13 or 14, wherein the N-acetyl glucosamine is in an amount of about 300 mg to about 900 mg in the composition.
16. The pharmaceutical composition of any one of clauses 13 to 15, further comprising one or more additional supplement agents.
17. The method according to clause 16, wherein the one or more additional supplement agents is a vitamin or an essential mineral.
18. The method according to clause 16 or 17, wherein the one or more additional supplement agents is vitamin A, a B vitamin, vitamin C. vitamin D, or zinc.
19. Use of N-acetyl glucosamine in the preparation of a medicament for preventing illness in a subject caused by influenza infection, a rhinovirus infection, or a betacoronavirus infection.
20. The use of clause 19, wherein the betacoronavirus infection is SARS-CoV-2 infection.
21. The use of clause 19 or 20, wherein N-acetyl glucosamine is in a prophylactically effective amount that prevents or reduces the chances of one or more symptoms in the subject, wherein the one or more symptoms is selected form the groups consisting of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.
22. The use of any one of clauses 19 to 21, wherein the medicament comprises an amount of N-acetyl glucosamine effective to prevent one or more symptoms of the betacoronavirus infection.
23. The use of any of one of clauses 19 to 22, wherein the medicament is administered intravenously, orally, subcutaneously, buccally, transdermally, or nasally.
24. The use of any of one of clauses 19 to 23, wherein the medicament is administered orally.
25. The use of any one of clauses 19 to 24, wherein the medicament comprises an amount of N-acetyl glucosamine in the range of about 200 mg to about 2100 mg.
26. The use of any one of clauses 19 to 25, wherein the medicament is administered once a day (QD), twice a day (BID), or three times a day (TID).
27. The use of any one of clauses 19 to 26, wherein the medicament is administered twice a day (BID), and the medicament comprises N-acetyl glucosamine in an amount of about 300 mg to about 900 mg in the medicament.
28. The use of any of clauses 19 to 27, wherein the medicament further comprises one or more additional supplement agents.
29. The use according to clause 28, wherein the one or more additional supplement agents is a vitamin or an essential mineral.
30. The use according to clause 28 or 29, wherein the one or more additional supplement agents is vitamin A, a B vitamin, vitamin C. vitamin D, or zinc.

### DETAILED DESCRIPTION

Before the present disclosure is further described, it is to be understood that this disclosure is not limited to particular embodiments described, and as such may, of course, vary. It is also to be understood that the terminology used herein is for the purpose of describing particular embodiments only, and is not intended to be limiting.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination.

### REPRESENTATIVE EMBODIMENTS

In some embodiments, the disclosure provides a method of preventing a subject from having a viral infection, such as influenza infection, rhinovirus infection, or betacoronavirus infection, comprising administering a prophylactically effective amount of N-acetyl glucosamine (NAG). In some embodiments, the disclosure provides the use of N-acetyl glucosamine in the preparation of a medicament for preventing a subject having a viral infection, such as influenza infection, rhinovirus infection, or betacoronavirus infection. In some embodiments, the disclosure provides a pharmaceutical composition comprising N-acetyl glucosamine, and optionally a pharmaceutically acceptable carrier or excipient, wherein the N-acetyl glucosamine is in a prophylactically effective amount for preventing a betacoronavirus infection. As used herein, the term “N-acetyl glucosamine” or “NAG” means 2-(acetylamino)-2-deoxy-β-D-glucopyranose (N-((2R,3R,4R,5S,6R)-2,4,5-trihydroxy-6-(hydroxymethyl)tetrahydro-2H-pyran-3-yl)acetamide), represented by the formula

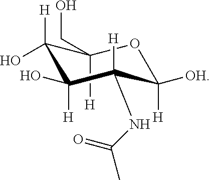

As used herein, the term “subject” refers to a human or, in the case of veterinary applications, can be a laboratory, agricultural, domestic, or wild animal. The methods described herein can be applied to subjects including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats.

As used herein, the term “prophylactically effective amount” refers to an amount of an agent (such as NAG) that elicits the biological or medicinal response in a subject (i.e. a tissue system, animal or human) that is being sought by a person, researcher, veterinarian, medical doctor or other clinician, which includes, but is not limited to, prevention of the symptoms of the disease or disorder associated with infection by a viral pathogen, prevention of infection by a viral pathogen, inhibition of the progression of viral infection, inhibition of the progression of symptoms associated with viral infection, prevention of the onset of symptoms associated with viral infection, prolonging negativity for viral infection of a subject that has tested negative for a virus, and prolonging the asymptomatic status of a subject that has tested positive for the presence of a virus. In some embodiments, the prophylactically effective amount is that amount of an agent (such as NAG) which may prevent or inhibit the progression of disease from asymptomatic to having symptoms of the disease at a reasonable benefit/risk ratio applicable to any medical prevention. In some embodiments, the prophylactically effective amount is that amount of an agent (such as NAG) which may prevent or inhibit the progression of symptoms associated with the viral infection at a reasonable benefit/risk ratio applicable to any medical prevention. In some embodiments, the prophylactically effective amount is that amount of an agent (such as NAG) which may reduce the chances of progression of symptoms associated with the viral infection at a reasonable benefit/risk ratio applicable to any medical prevention. It will be appreciated that the viral pathogen or viral infection is in reference to viruses, such as influenza viruses, rhinoviruses, and betacoronaviruses, including SARS-CoV-2, and viral infection, such as influenza infection, rhinovirus infection, or betacoronavirus infection.

It will be appreciated that the methods, uses, compositions, or compounds described herein can be applied to illnesses resulting from a variety of viral infections, including but not limited to, influenza, coronavirus, and rhinovirus, and the like. In some embodiments, the methods, uses, compositions, or compounds described herein can be applied to prevention of illnesses resulting from influenza, rhinovirus, or coronaviruses, especially betacoronaviruses, which can affect humans and can cause respiratory tract infections that range from mild to lethal. In some embodiments, the betacoronavirus include, but are not limited to, SARS-COV-1 (SARS), MERS-CoV (MERS), HCoV-HKU1, HCoV-NL63, HCoV-OC43, HCoV-229E, SARS-CoV-2 (COVID-19), and the like.

As used herein, “COVID-19” refers to coronavirus disease 2019, caused by the SARS-CoV-2 coronavirus. It will be appreciated that the SARS-CoV-2 coronavirus has been found to have a number of lineages (clades), such as S, O, L, V, G, GH, GR, GV, and the like, variants, such as B.1.1.207, B.1.1.248, variant of concern 202012/01, Cluster 5, 501.VA variant, 501Y.V2 variant, CAL.20C. and the like, and mutations, such as D614G, E484K, N501Y, and the like, and that the methods, uses, compositions, or compounds described herein can be used for the prevention of COVID-19 in any one or more of its lineages, variants, or mutations. It will be appreciated that populations of pathogenic cells that cause inflammation, for example, as a result of SARS-CoV-2 infection resulting in COVID-19, can lead to a variety of illnesses and symptoms in a subject, as described herein, such as pneumonia.

In some embodiments, the illnesses or symptoms a subject may experience as a result of influenza infection, rhinovirus infection, or coronavirus, especially SARS-CoV-2 (COVID-19), infection include, but are not limited to, pneumonia, Acute Respiratory Distress Syndrome (ARDS), systemic inflammatory response syndrome, such as cytokine release syndrome (CRS), a central nervous system disorder, inflammation, multisystem inflammatory syndrome, vasculitis, fever, fever with rigors, fatigue, anorexia, myalgias, arthralgias, nausea, vomiting, headache, rash, kidney disease, intestinal disease, liver disease, diarrhea, tachypnea, hypoxemia, tachycardia, widened pulse pressure, hypotension, increased cardia output, potentially diminished cardiac output, Deep Vein Thrombosis (DVT), microthrombosis, endotheliopathy and blood clotting disorders leading to thrombosis (i.e. Ischemic Stroke), elevated blood glucose levels, elevated D-dimer, hypofibrinogenemia, hypofibrinogenemia with bleeding, azotemia, transaminitis, hyperbilirubinemia, mental state changes, confusion, delirium, word finding difficulty, hallucinations, tremor, dysmetria, altered gait, and seizures. In some embodiments, the illnesses or symptoms a subject may experience from coronavirus infection, especially SARS-CoV-2 (COVID-19), infection include, but are not limited to, Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), microthrombosis, endotheliopathy and blood clotting disorders leading to thrombosis (i.e. Ischemic Stroke), and elevated blood glucose levels.

In some instances, the illness and/or symptoms a subject may experience as a result of influenza infection, rhinovirus infection, or coronavirus, including SARS-CoV-2 (COVID-19), infection can lead to intubation or mechanical ventilation or death. Without being bound by theory, it is believed the methods, uses, compositions, or compounds described herein decreases viral RNA replication by interaction with the glucosamine receptor, which can lead to decreased viral loads, and ultimately leading to a lower incidence of infection or progression from asymptomatic to symptomatic leading to possible intubation or mechanical ventilation or death.

It will be appreciated that the methods, uses, compositions, or compounds described herein can be administered in any of the modes of administration known in the art. As used herein, “administering” or “administered” includes all means of introducing the compounds and compositions described herein to a subject, including, but are not limited to, oral (po), intravenous (iv), intramuscular (im), subcutaneous (sc), transdermal, inhalation, buccal, ocular, sublingual, nasal, vaginal, rectal, and the like. The methods, uses, compositions, or compounds described herein may be administered in unit dosage forms and/or formulations containing conventional nontoxic pharmaceutically-acceptable carriers, adjuvants, and/or vehicles.

In some embodiments, the methods, uses, compositions, or compounds described herein can be administered orally. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Solid formulations for oral administration may be formulated to be immediate and/or modified release formulations. Modified release formulations include delayed, sustained, pulsed, controlled, targeted and programmed release formulations.

In some embodiments, the methods, uses, compositions, or compounds described herein can be administered directly into the blood stream, into muscle, or into an internal organ. Suitable means for parenteral administration include intravenous, intraarterial, intraperitoneal, intrathecal, intraventricular, intraurethral, intrastemal, intracranial, intramuscular and subcutaneous means of administration.

In some embodiments, the methods, uses, compositions, or compounds described herein can be co-administered or co-formulated with one or more additional supplemental agents, such as vitamins, essential minerals, drugs, and the like. In some embodiments, the one or more additional supplement agents is vitamin A, a B vitamin, such as folate, vitamin C. vitamin D, or zinc. In some embodiments, NAG can be co-administered or co-formulated with between about 200 mg and about 2000 mg of vitamin C. In some embodiments, NAG can be co-administered or co-formulated with between about 500 mg and about 1500 mg of vitamin C. In some embodiments, NAG can be co-administered or co-formulated with between about 750 mg and about 1250 mg of vitamin C. In some embodiments, NAG can be co-administered or co-formulated with between about 900 mg and about 1100 mg of vitamin C. In some embodiments, NAG can be co-administered or co-formulated with between about 50 mcg and about 500 mcg of folate. In some embodiments, NAG can be co-administered or co-formulated with between about 100 mcg and about 200 mcg of folate. In some embodiments, NAG can be co-administered or co-formulated with between about 100 mcg and about 150 mcg of folate. In some embodiments, NAG can be co-administered or co-formulated with between about 20 mg and about 100 mg of zinc. In some embodiments, NAG can be co-administered or co-formulated with between about 40 mg and about 75 mg of zinc. In some embodiments, NAG can be co-administered or co-formulated with between about 40 mg and about 60 mg of zinc.

In some embodiments, NAG can be co-administered or co-formulated with between about 200 mg and about 2000 mg of vitamin C, and with between about 50 mcg and about 500 mcg of folate. In some embodiments, NAG can be co-administered or co-formulated with between about 750 mg and about 1250 mg of vitamin C, and with between about 100 mcg and about 200 mcg of folate. In some embodiments, NAG can be co-administered or co-formulated with between about 900 mg and about 1100 mg of vitamin C, and with between about 100 mcg and about 150 mcg of folate. In some embodiments, NAG can be co-administered or co-formulated with between about 200 mg and about 2000 mg of vitamin C, and with between about 100 mcg and about 150 mcg of folate. In some embodiments, NAG can be co-administered or co-formulated with between about 750 mg and about 1250 mg of vitamin C, and with between about 100 mcg and about 150 mcg of folate. In some embodiments, NAG can be co-administered or co-formulated with between about 200 mg and about 2000 mg of vitamin C, with between about 100 mcg and about 150 mcg of folate, and with between about 20 mg and about 100 mg of zinc. In some embodiments, NAG can be co-administered or co-formulated with between about 750 mg and about 1250 mg of vitamin C, with between about 100 mcg and about 150 mcg of folate, and with between about 20 mg and about 100 mg of zinc. In some embodiments, NAG can be co-administered or co-formulated with between about 900 mg and about 1100 mg of vitamin C, with between about 100 mcg and about 150 mcg of folate, and with between about 20 mg and about 100 mg of zinc.

Any effective regimen for administering the compounds and compositions described herein can be used. For example, compounds and compositions described herein can be administered as single doses, or the doses can be divided and administered as a multiple-dose daily regimen. Further, a staggered regimen, for example, one to five days per week can be used as an alternative to daily prevention. In some embodiments, a subject is administered multiple doses in the methods, uses, compounds, or compositions described herein. In some embodiments, a subjected is administered multiple doses (preferably about 2 up to about 80 doses) with a compound or composition as described herein, for example, at 8-72 hour intervals or at 8-12 hour intervals.

Any suitable course of administration with the N-acetyl glucosamine as described herein can be used. In one embodiment, individual doses and dosage regimens are selected to provide a total dose administered during a given day of about 200 mg to about 2100 mg; or about 500 mg to about 1500 mg. In one embodiment, individual doses and dosage regimens are selected to provide a total dose administered during a given day of about 200 mg to about 2100 mg; or about 500 mg to about 1500 mg, co-administered or co-formulated with one or more additional supplemental agents as described herein, such as vitamin C, folate, zinc, and the like, as described herein. In some embodiments, the N-acetyl glucosamine is administered in the methods or uses described herein in a single daily dose (QD), or in a twice daily dose (BID), or a three times daily dose (TID). In some embodiments, the N-acetyl glucosamine is administered in the methods or uses described herein in a twice daily dose (BID) at a dose of about 300 mg to about 900 mg per dose. In some embodiments, the N-acetyl glucosamine is administered in the methods or uses described herein in a twice daily dose (BID) at a dose of about 300 mg to about 900 mg per dose, co-administered or co-formulated with one or more additional supplemental agents as described herein, such as vitamin C, folate, zinc, and the like, as described herein. In some embodiments, the N-acetyl glucosamine is administered in the methods or uses described herein in cycles lasting days a week, 2 weeks, 3 weeks, 4 weeks, 5 weeks, and the like. In some embodiments, the N-acetyl glucosamine is administered daily in the methods or uses described herein for between 10 and 300 days, or until cessation of administration when confidence in prevention is achieved. In some embodiments, the N-acetyl glucosamine is administered daily in the methods or uses described herein for between 10 and 200 days, or until cessation of administration when confidence in prevention is achieved. In some embodiments, the N-acetyl glucosamine is administered daily in the methods or uses described herein for between 25 and 100 days, or until cessation of administration when confidence in prevention is achieved. In some embodiments, the N-acetyl glucosamine is administered daily in the methods or uses described herein for about 10 to 30 days, or until cessation of administration when confidence in prevention is achieved.

It will be appreciated that the unitary daily dosage of the N-acetyl glucosamine described herein can vary significantly depending on the patient condition, the virus being prevented, the route of administration of the N-acetyl glucosamine, and the possibility of co-administration of additional supplemental agents, as described herein. The effective amount to be administered to a patient is based on body surface area, mass, and physician assessment of patient condition.

### EXAMPLE 1

To assess the use of N-acetyl glucosamine alone or in combination with one or more additional supplemental agents, such as vitamins or essential minerals, a physician will examine and test a subject (or patient) in a clinical setting. including the use of standard clinical assessments or available diagnostic tests, including tests such as mRNA, PCR, antibody, and the like. Only subject who test positive for covid-19 and are asymptomatic will be enrolled into the study for this example.

The subject population will be the general population regardless of age, sex, race, or ethnic origin, and may include subjects having pre-existing conditions such as obesity, diabetes, heart disease, autoimmune disorders, or may be otherwise immune compromised.

Upon identification as a subject through a positive diagnostic test for covid-19, the subject will be recommended for study of the prevention of covid-19 with N-acetyl glucosamine. The subject will be orally administered a composition including at least N-acetyl glucosamine, and optionally one or more supplemental agents, such as vitamins or essential minerals, at a dose (such as 500 mg, 600 mg, 700 mg, 800 mg, 900 mg, or 1000 mg) twice daily (BID dosing) for 14-30 days. The subject may be assessed for clinical status by standard clinical assessments or available diagnostic tests at interim periods as determined by the preventing physician prior to the end of the prevention cycle. At the determination of the preventing physician, the subject may be co-administered one or more additional supplemental agents, including vitamin C, folate and/or zinc at a standard dose, as described herein, and at an interval to be determined by the preventing physician that can be either at the same time, before, or after administration of N-acetyl glucosamine. The results of the prevention study will be collected and compared to a control arm of individuals who test positive for covid-19 using available diagnostic tests, including tests such as mRNA, PCR, antibody, and the like. The results will be bench-marked against the control arm by metrics including but not limited to monitoring for symptoms such as those described herein for covid-19.

1. A method of preventing illness caused by a viral infection, such as influenza infection, rhinovirus infection, or betacoronavirus infection, comprising administering a prophylactically effective amount of N-acetyl glucosamine.

2. The method of claim 1, wherein the betacoronavirus infection is SARS-CoV-2 infection.

3. The method of claim 1, wherein the administering a prophylactically effective amount of N-acetyl glucosamine prevents or reduces the chances of one or more symptoms in the subject, wherein the one or more symptoms is selected form the groups consisting of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

4. The method of claim 1, wherein the prophylactically effective amount prevents one or more symptoms of the betacoronavirus infection.

5. The method of claim 1, wherein the N-acetyl glucosamine is administered intravenously, orally, subcutaneously, buccally, transdermally, or nasally.

6. The method of claim 1, wherein the N-acetyl glucosamine is administered orally.

7. The method of claim 1, wherein the prophylactically effective amount of the N-acetyl glucosamine is in the range of about 200 mg to about 2100 mg.

8. The method of claim 1, wherein the prophylactically effective amount of the N-acetyl glucosamine is administered once a day (QD), twice a day (BID), or three times a day (TID).

9. The method of claim 1, wherein the prophylactically effective amount of the N-acetyl glucosamine is administered twice a day (BID), at a dose of about 300 mg to about 900 mg per dose.

10. The method of claim 1, further comprising administration of one or more additional supplement agents.

11. The method according to claim 10, wherein the one or more additional supplement agents is a vitamin or an essential mineral.

12. The method according to claim 10, wherein the one or more additional supplement agents is vitamin A, a B vitamin, vitamin C. vitamin D, folate, or zinc.

13. A pharmaceutical composition comprising N-acetyl glucosamine, and optionally a pharmaceutically acceptable carrier or excipient, wherein the N-acetyl glucosamine is in a prophylactically effective amount for preventing an influenza, a rhinovirus, or a betacoronavirus infection.

14. The pharmaceutical composition of claim 13, wherein the N-acetyl glucosamine is in an amount of about 200 mg to about 2100 mg in the composition.

15. The pharmaceutical composition of claim 13, wherein the N-acetyl glucosamine is in an amount of about 300 mg to about 900 mg in the composition.

16. The pharmaceutical composition of claim 13, further comprising one or more additional supplement agents.

17. The composition according to claim 16, wherein the one or more additional supplement agents is a vitamin or an essential mineral.

18. The composition according to claim 16, wherein the one or more additional supplement agents is vitamin A, a B vitamin, vitamin C. vitamin D, folate, or zinc.

19-30. (canceled)
